(19) United States
(12) Patent Application Publication
(10) Pub. No.: US 2010/0168506 A1
(43) Pub. Date: Jul. 1, 2010

(54) Method For Preparing Drug-Eluting Stent Having Nano-Structured Pattern

(75) Inventors: Myoung-Woon Moon, Seoul (KR); Kwang Ryeol Lee, Seoul (KR); Dong Keun Han, Seoul (KR); Kwi- Deok Park, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Korea Institute of Science and Technology, Seoul (KR)

(21) Appl. No.: 12/368,537

(22) Filed: Feb. 10, 2009

(30) Foreign Application Priority Data
Dec. 29, 2008 (KR) 10-2008-0135808

(51) Int. Cl. A61F 2/04; A61F 2/06

(52) U.S. Cl. 600/36; 623/1.15

(57) ABSTRACT

This invention relates to a method for preparing a drug-eluting stent using a chemical vapor deposition, the method comprising modifying the surface of a biodegradable polymer with nanostructures through a plasma-assisted chemical vapor deposition so as to improve drug-loading capability and drug elution rate.

### FIELD OF THE INVENTION

The present invention relates to a method for preparing a drug-eluting stent using a chemical vapor deposition.

### BACKGROUND OF THE INVENTION

Recently, with the population ageing, the demand for vascular implants such as vascular stents for coronary and peripheral arteries is increasing, and thus the importation of these products is also constantly increasing. However, vascular stents can cause acute occlusion due to thrombus formation after implantation, and the stents themselves act as traumatic factors in vascular walls to induce intimal hyperplasia, thus causing a problem of restenosis. For this reason, functional surface modification technology having a drug-eluting function of delivering therapeutic drugs directly into blood vessels has been required together with surface treatment for inhibiting thrombosis. Thus, Hepacoat Corporation et al. has commercially marketed a stent coated with heparin for inhibiting thrombosis, and Cordis Corporation has commercially marketed Cypher™ as a first drug-eluting stent for preventing vascular restenosis. However, problems in that patients implanted with these stents have died recently occurred, and thus the need to develop a stent having improved drug-eluting performance exists.

Meanwhile, studies on diamond-like carbon (DLC) coatings and the activation thereof are being studied. Such coatings are biocompatible on the surface of a material, such as nitinol (TiNi) or stainless steel that is the main material of a general alloy stent loaded with no drug. Also, these coatings protect vascular walls implanted with stents and, in addition, can prevent thrombosis and restenosis. Particularly, various methods for synthesizing amorphous hard carbon films have been developed, and a coated stent is currently being marketed by Phytis AG. Moreover, US Patent Publication No. 2006/0200231 discloses a method for preparing a drug-eluting stent (DES), in which a carbon-containing material and DLC are used and a metal bar is inserted to improve the porosity.

Also, as technologies for imparting biocompatibility, there have been attempts to improve biocompatibility by surface modification with microstructures or nanostructures. For example, methods of modifying the surfaces of materials using an ion beam or plasma were discovered in the first half of the 1990s and were significantly improved through the latter half of the 1990s. However, in most cases, a glassy metal, an amorphous material such as amorphous silicon (a-Si), or a crystalline material such as silicon (Si) were used as materials exposed to ion beams.

Finkelstein et al. attempted to control the kind and release rate of drugs by forming a deep groove in the surface of a metal stent and inserting into the groove a polymer loaded with 2-3 kinds of drugs in multiple layers (Finkelstein et al., Circulation, 107 (2003) 777-784). Also, Ankur Raval et al. attempted to control a drug-loading function by depositing a biodegradable polymer and a non-biodegradable polymer on the surface of a stent in four layers (Ankur Raval, Trends Biomater. Artif. Organs, Vol. 20(2) (2007) 101-110).

However, there has been no case in which the surface of polymers currently being widely used in biological research, for example, a polymer such as (polylactic-co-glycolic acid PLGA), has been modified using ion beams. Furthermore, a study on the formation of a nanostructured pattern for loading drugs on the polymer surface has not yet been conducted.

### SUMMARY OF THE INVENTION

Accordingly, it is an object of the present invention to provide a method for preparing a drug-eluting stent having a nanostructured pattern using a plasma-assisted plasma vapor deposition.

In accordance with one aspect of the present invention, there is provided a method for preparing a drug-eluting stent, comprising the steps of: (a) forming a first biodegradable polymer layer on the surface of a stent; (b) forming a nanostructured pattern on the surface of the first biodegradable polymer layer by treatment with ion beams or plasma using a plasma-assisted chemical vapor deposition (PACVD); and, optionally, (c) forming a second biodegradable polymer layer on the first biodegradable polymer layer having the nanostructured pattern formed thereon, at least one of the first and second biodegradable polymer layers being loaded with identical or different drugs.

The inventive method for preparing a stent using a plasma-assisted chemical vapor deposition can improve drug-loading capability and control drug elution rate by modifying the surface of a biodegradable polymer with nanostructures. Thus, the method of the present invention is useful for the preparation of a drug-eluting stent.

### BRIEF DESCRIPTION OF THE DRAWINGS

The above and other objects and features of the present invention will become apparent from the following description of the invention, when taken in conjunction with the accompanying drawings, which respectively show:

FIG. 1 shows a schematic diagram showing a process for preparing the drug-eluting stent of the present invention;

FIG. 2a shows an optical micrograph of a portion of the drug-eluting stent prepared in Example 1;

FIG. 2b shows the cross section of a drug-eluting stent and a scanning electron microscope photograph of surface of the drug-eluting stent prepared in Example 1; and

FIG. 3a to 3c show scanning electron microscope photographs of a biodegradable polymer surface before plasma treatment, a biodegradable polymer surface after plasma treatment at a bias voltage of −800 V and a drug-loaded biodegradable polymer surface after plasma treatment according to the present invention, respectively.

### DETAILED DESCRIPTION OF THE INVENTION

The inventive method for preparing a drug-eluting stent comprises: (a) forming a first biodegradable polymer layer on the surface of a stent; (b) forming a nanostructured pattern on the surface of the first biodegradable polymer layer by treatment with ion beams or plasma using a plasma-assisted chemical vapor deposition (PACVD); and, optionally, (c) forming a second biodegradable polymer layer on the first biodegradable polymer layer having the nanostructured pattern formed thereon, at least one of the first and second biodegradable polymer layers being loaded with identical or different drugs.

In the inventive method, the first biodegradable polymer layer may be loaded with a drug by loading a drug into the first biodegradable polymer layer having the nanostructured pattern formed thereon, obtained from step (b). Also, the first biodegradable polymer layer may be loaded with a drug by coating a drug-loaded biodegradable polymer on the surface of the stent, in step (a). Herein, the first biodegradable polymer layer may be further loaded with a drug by loading a second drug into the first biodegradable polymer layer having the nanostructured pattern formed thereon, obtained from step (b).

In the inventive method, the second biodegradable polymer layer may be loaded with a drug by coating a drug-loaded biodegradable polymer on the first biodegradable polymer layer having the nanostructured pattern formed thereon, in step (c). Herein, the first biodegradable polymer layer may also be loaded with at least one drug in the same manner as described in the preceding paragraph.

The inventive method for preparing the drug-eluting stent will now be described in further detail.

In step (a) or (c), the first and second biodegradable polymer layers can be formed by coating on the stent surface to a thickness of 10-20 μm using a spraying method (Chen et al., J of controlled release, 108:178-189, 2005). Herein, the biodegradable polymer may be a drug-loaded biodegradable polymer.

The stent may be made of a material which is conventionally used as a material for stents, such as stainless steel or nitinol (NiTi), and the thickness thereof may vary as occasion demands.

The biodegradable polymer may be a polymer having excellent biodegradability and biocompatibility and is preferably selected from the group consisting of polyglycolic acid (PGA), poly-L-lactic acid (PLLA), poly-DL-lactic acid (PDLLA), poly(lactic acid-co-glycolic acid) (PLGA), poly-ε-caprolactone (PCL), polyamino acid, polyanhydride, polyorthoester, and copolymers thereof.

In step (b), the nanostructured surface is formed on the surface of the biodegradable polymer, coated on the surface of the stent in step (a), by treatment with ion beams or plasma using a plasma-assisted chemical vapor deposition (PACVD).

The ion beam or plasma treatment can be carried out using a material selected from the group consisting of argon (Ar), nitrogen (N₂), oxygen (O₂), tetrafluoromethane (CF₄), and mixtures thereof. Also, the ion beam or plasma treatment can be carried out at a voltage ranging from −100 V to −100 kV, preferably from −500 V to −1000 V, at a power ranging from 1 W to 10 kW, preferably from 100 W to 500 W, for a time ranging from 1 second to 2 hours, preferably 1 minute to 10 minutes.

The first biodegradable polymer layer subjected to the above-described ion beam or plasma treatment has a nanostructured pattern formed thereon, and the pattern may be nano-hole, nano-wrinkle, nano-hair or nano-network. The nanostructured pattern can have a width ranging from 200 nm to 1 μm, preferably 200 nm, and a height ranging from 100 nm to 500 nm, preferably 200 nm. The width and height of the nanostructured pattern on the surface may vary depending on various conditions.

The nanostructured pattern formed in step (b) can increase the bonding strength between the biodegradable polymer and the metal stent and improve drug-loading capability and drug elution rate.

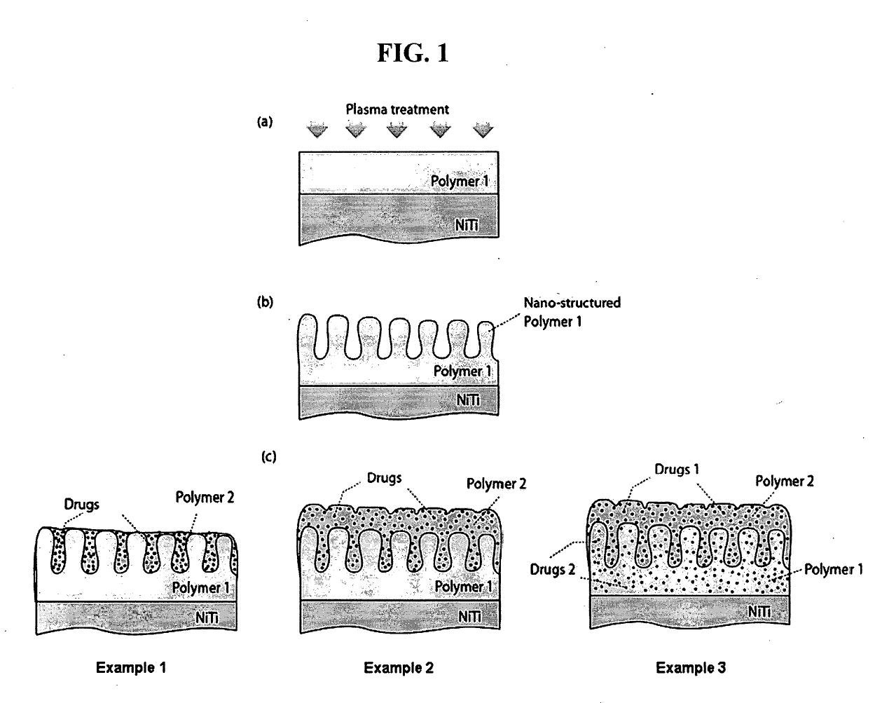

FIG. 1 is a schematic diagram showing a process of preparing a stent according to the embodiments of the present invention.

### EXAMPLES

Hereinafter, the present invention will be described in further detail with reference to examples. It is to be understood, however, that these examples are for illustrative purposes only and the scope of the present invention is not limited only to these examples.

### Example 1

### 1-1. Coating with Biodegradable Polymer

Poly(lactic acid-co-glycolic acid) (PLGA; Boehringer Ingelheim AG, Germany) (biodegradable polymer 1) was dissolved in methylene chloride (CH₂Cl₂) at a concentration of 10 wt %, and then coated on the surface of a laser-processed stent (Taewoong Medical Co., Ltd., Korea) to a thickness of 10 μm by a conventional spraying method (Chen et al., J of controlled release, 108:178-189, 2005).

### 1-2. Treatment with Ion Beams or Plasma

The surface of the PLGA-coated stent, obtained in Example 1-1, was treated with argon plasma at a radio frequency of 13.56 MHz using a plasma-assisted chemical vapor deposition (PACVD). Specifically, the surface of the PLGA polymer layer was treated with argon (Ar) plasma at a chamber pressure of 1.33 Pa at a voltage of −800 V for 5 minutes, thus forming a nano-wrinkle structure. Herein, the structure may somewhat change depending on the treatment time and the chamber pressure.

### 1-3. Coating with Drug-Loaded Biodegradable Polymer

PLGA (Boehringer Ingelheim AG, Germany) (biodegradable polymer 2) was dissolved in methylene chloride at a concentration of 3 wt %, and then paclitaxel (Aldrich, 50 mg) (drug 1) was added and mixed therewith in an amount corresponding to 1/10 of PLGA. The mixed solution was coated on the surface of the biodegradable polymer layer having the nanostructured pattern formed thereon, obtained in Example 1-2, to a thickness of 2 μm using the same spraying method as in Example 1-1.

### Example 2

This Example was carried out in the same manner as in Example 1, except that, in Example 1-3, the biodegradable polymer 2 was coated to a thickness of 10 μm on the surface of the biodegradable polymer 1 having the nanostructured pattern formed thereon.

### Example 3

This Example was carried out in the same manner as in Example 1, except that, in the biodegradable polymer-coating step of Example 1-1, PLGA (Boehringer Ingelheim AG, Germany) (biodegradable polymer 1) was dissolved in toluene at a concentration of 10 wt %, and then paclitaxel (Aldrich, 50 mg) (drug 2) was added thereto, thereby coating on the surface of a laser-processed stent (Taewoong Medical Co., Ltd., Korea) to a thickness of 10 μm by a conventional spraying method.

### Experimental Example 1

### Analysis of Surface

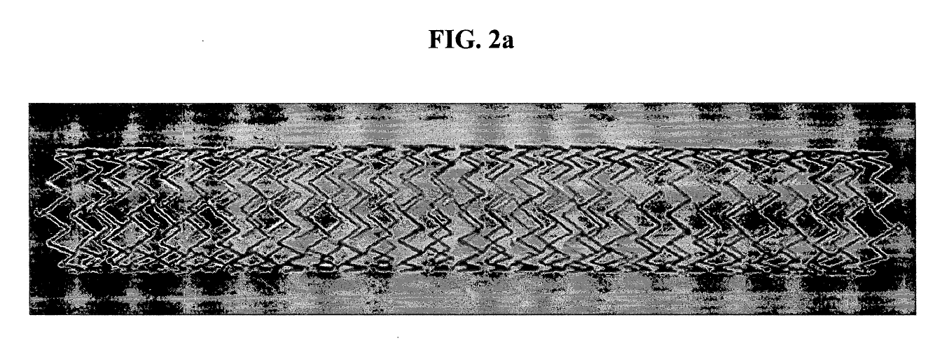

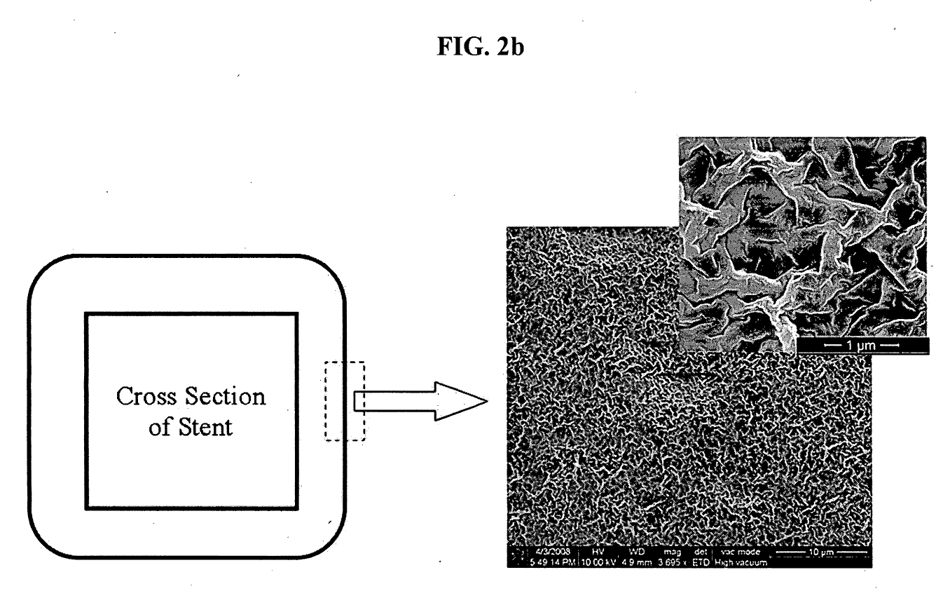

To analyze the surface of the stent obtained in Example 1, an atomic force microscope (AFM) (AutoProbe CP Research System, Thermo Microscope Inc., USA) was used, and the surface roughness of a 2 μm×2 μm region was measured in a non-contact mode. The surface roughness was measured as a Root-Mean-Square (RMS). Also, the morphology of the surface was photographed with a scanning electron microscope (nano-SEM, FEI Inc.), and the results are shown in FIGS. 2a and 2b. FIG. 2a shows an optical microscopic image of a portion of the NiTi stent, and FIG. 2b shows the cross section of the stent and a scanning electron microscope photograph of surface thereof. As can be seen in FIG. 2b, a nanostructured pattern was formed on the surface of the stent. The surface nanostructured pattern had a width of about 200 nm and a height of about 100 nm. The width and height of the surface nanostructured pattern may vary depending on various conditions.

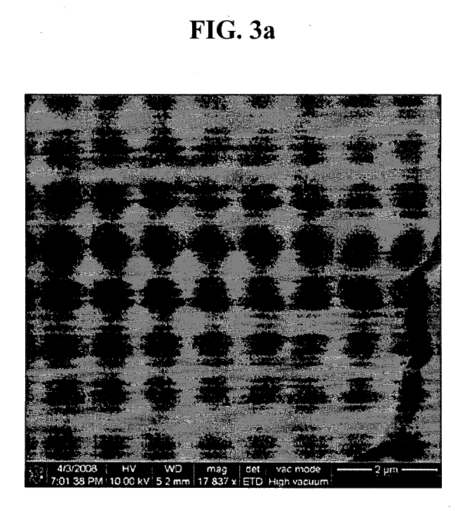

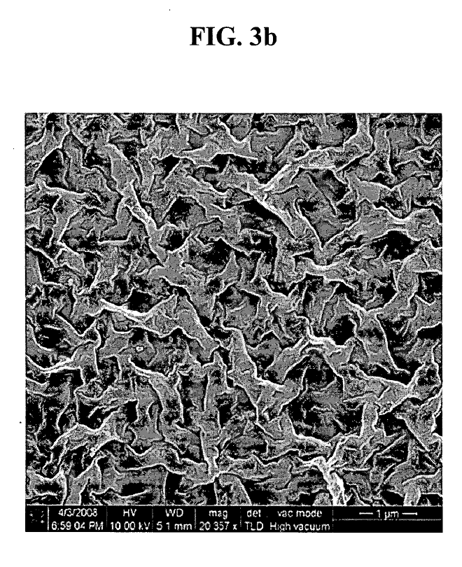

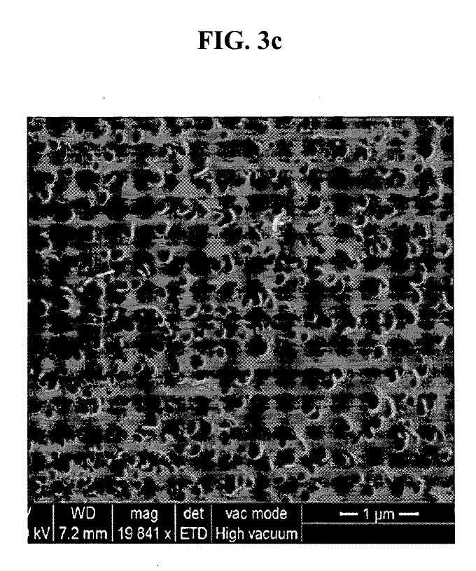

Also, a biodegradable polymer surface before plasma treatment, a biodegradable polymer surface after plasma treatment at a bias voltage of −800 V and a drug-loaded biodegradable polymer surface after plasma treatment according to the present invention were photographed with a scanning electron microscope (nano-SEM, FEI Inc.), and the results are shown in FIG. 3a to 3c. As can be seen in FIG. 3a to 3c, as compared to the smooth surface before plasma treatment (FIG. 3a), nano-sized patterns (FIG. 3b) and nano-sized holes (FIG. 3c) were formed by the plasma treatment.

While the invention has been described with respect to the above specific embodiments, it should be recognized that various modifications and changes may be made to the invention by those skilled in the art which also fall within the scope of the invention as defined by the appended claims.

What is claimed is:

1. A method for preparing a drug-eluting stent, comprising the steps of:
(a) forming a first biodegradable polymer layer on the surface of a stent;
(b) forming a nanostructured pattern on the surface of the first biodegradable polymer layer by treatment with ion beams or plasma using a plasma-assisted chemical vapor deposition (PACVD); and, optionally,
(c) forming a second biodegradable polymer layer on the first biodegradable polymer layer having the nanostructured pattern formed thereon,
at least one of the first and second biodegradable polymer layers being loaded with identical or different drugs.

2. The method of claim 1, wherein the first biodegradable polymer layer is loaded with a drug by loading a drug into the first biodegradable polymer layer having the nanostructured pattern formed thereon, obtained from step (b).

3. The method of claim 1, wherein the first biodegradable polymer layer is loaded with a drug by coating a drug-loaded biodegradable polymer on the surface of the stent, in step (a).

4. The method of claim 3, wherein the first biodegradable polymer layer is further loaded with a drug by loading a second drug into the first biodegradable polymer layer having the nanostructured pattern formed thereon, obtained from step (b).

5. The method of claim 1, wherein the second biodegradable polymer layer is loaded with a drug by coating a drug-loaded biodegradable polymer on the surface of the first biodegradable polymer layer, in step (c).

6. The method of claim 5, wherein the first biodegradable polymer layer is loaded with a drug by loading a drug into the first biodegradable polymer layer having the nanostructured pattern formed thereon, obtained from step (b).

7. The method of claim 5, wherein the first biodegradable polymer layer is loaded with a drug by coating a drug-loaded biodegradable polymer on the surface of the stent, in step (a).

8. The method of claim 7, wherein the first biodegradable polymer layer is further loaded with a drug by loading a second drug into the first biodegradable polymer layer having the nanostructured pattern formed thereon, obtained from step (b).

9. The method of claim 1, wherein the first and second biodegradable polymer layers are formed by coating with a biodegradable polymer selected from the group consisting of polyglycolic acid (PGA), poly-L-lactic acid (PLLA), poly-DL-lactic acid (PDLLA), poly(lactic acid-co-glycolic acid) (PLGA), poly-ε-caprolactone (PCL), polyamino acid, polyanhydride, polyorthoester, and copolymers thereof.

10. The method of claim 1, wherein the ion beam or plasma treatment in step (b) is carried out using a material selected from the group consisting of argon (Ar), nitrogen (N₂), oxygen (O₂), tetrafluoromethane (CF₄), and mixtures thereof.

11. The method of claim 1, wherein the ion beam or plasma treatment in step (b) is carried out at a voltage ranging from −100 V to −100 kV.

12. The method of claim 1, wherein the ion beam or plasma treatment in step (b) is carried out at a power ranging from 1 W to 10 kW.

13. The method of claim 1, wherein the ion beam or plasma treatment in step (b) is carried out for a time ranging from 1 second to 2 hours.

14. The method of claim 1, wherein the nanostructured pattern in step (b) is selected from the group consisting of nano-hole, nano-wrinkle, nano-hair and nano-network.
